(19) United States
(12) Patent Application Publication
(10) Pub. No.: US 2022/0079791 A1
(43) Pub. Date: Mar. 17, 2022

(54) GASTROINTESTINAL IMPLANT AND POSITIONING APPARATUS THEREFOR

(71) Applicants: Alexander BUTZ, Vienna (AT); Viktor GRABLOWITZ, Vienna (AT)

(72) Inventors: Alexander BUTZ, Vienna (AT); Viktor GRABLOWITZ, Vienna (AT)

(21) Appl. No.: 17/420,256

(22) PCT Filed: Jan. 3, 2019
(86) PCT No.: PCT/EP2019/050112

§ 371 Date: Jul. 1, 2021

(51) Int. Cl. A61F 5/00

(52) U.S. Cl. CPC A61F 5/0079; A61F 5/005; A61F 5/0089

(57) ABSTRACT

A gastrointestinal implant comprising a tube-shaped element, embodied to transport chyme through at least one section of the human intestine and a first fastener connected to the tube-shaped element and having a first contact surface. The first fastener is embodied to be positioned in a section of the duodenum adjacent to the pylorus. A second fastener connected to the tube-shaped element or to the first fastener has a second contact surface. The second fastener is embodied to be positioned in the gastric antrum. A connecting element connects the first fastener or the tube-shaped element to the second fastener. The connecting element is embodied such that the first and second fasteners are pressed against one another with the first and second contact surface thereof, respectively, in the position of the fasteners in the duodenum and in the gastric antrum, respectively, with the pylorus positioned therebetween, without the fasteners being lifted off of the pylorus.

### FIELD OF THE INVENTION

The present invention concerns the field of obesity and the related sequelae such as diabetes mellitus, arterial hypertension and the accompanying increased risks of stroke, heart attack and cancer, as well as sequelae of the spinal column and musculoskeletal system.

Frequently, many patients are not capable of fighting their obesity by their own efforts, for example, by eating less or more healthily. Often times, it is also not possible for patients to provide the movement that is so necessary for fighting obesity, since the obesity is often already so far advanced that the aforementioned sequelae of the spinal column and musculoskeletal system cause such severe pain that adequate movement is not possible.

These patients therefore require additional aid that allow the excess weight to be rapidly reduced to such a level that at least pain-free movement is once again possible. However, in this case, the accompanying enhanced wellness very often also motivates patients to make further efforts to reduce their weight, so that this initial weight-reduction aid helps the patients to achieve the additional weight reduction on their own or to at least maintain the weight which has been reduced to a less harmful level.

### PRIOR ART

In the medical field, various methods are known for enabling the described rapid weight reduction. For example, performing stomach reductions or creating gastric bypasses is known. Since this involves a difficult, invasive operation on the human body, this method is not only technically very challenging, but is also associated with a risk that accompanies every invasive operation and which, among other things, also includes the risk accompanying the required longer anesthesia time.

In the search for alternative, less risky methods, there has been a shift to positioning a plastic tube and fastening means (gastrointestinal implant) starting in the human small intestine, in particular the duodenum, and in this manner partially deactivating the absorption of food. The supplied food is not absorbed in the region of the plastic tube, since the plastic tube internally lines the intestinal lumen and thus prevents contact with the intestinal wall and therefore the absorption of nutrients. With a corresponding placement of the plastic tube in the small intestine (duodenum), in direct connection with the pylorus, the plastic tube also causes the digestive enzymes (bile and pancreatic secretion) to not come into contact with the food until after the end of the plastic tube, whereby the food conversion is additionally limited and duodenogastric reflux is prevented.

The length of the tube can thereby be individually selected and influences the speed at which and the maximum extent to which the weight reduction is to proceed. Weight reductions of >70% in one year are possible. In addition, as one would expect, diabetes mellitus type II is already in remission in 80% of patients after 3 months, as has been established in various studies and is known from bariatric surgery. Arterial hypertension, cardiac arrhythmia, musculo skeletal diseases, and other comorbidities also markedly improve after a weight reduction of this type, and are even in remission.

Bringing the gastrointestinal implant into position in the small intestine endoscopically, and also removing it from said position again, is known. Therefore, no operation is necessary. The method is non-invasive, and therefore involves no scarring and is fully reversible. The application of the tube takes place under sedation or short-acting anesthetics.

To allow the performance of the endoscopic positioning of the gastrointestinal implant in the small intestine, a positioning apparatus is required on the one hand, which apparatus initially protects the gastrointestinal implant during the introduction into the small intestine and then enables a securing and unfolding of the plastic tube, while on the other hand suitable fastening means are also required which enable a reliable, essentially immovable securing of the plastic tube in the duodenum.

Primarily, the securing of the plastic tube and its unfolding in the small intestine thereby continue to cause problems.

A proper positioning and securing of the plastic tube is imperative for the success of this non-invasive method. It must be possible to guarantee that a displacement of the plastic tube caused by food intake or peristalsis does not occur, as a result of which sections of the small intestine, in particular the duodenum, could become exposed and food could be absorbed in said section. Furthermore, it must be ensured that no food can reach the space between the plastic tube and the wall of the small intestine, which would also cause food to be absorbed.

From WO2011/062882A1, a system is known which enables the endoscopic positioning of the plastic tube in the small intestine. The system according to a first embodiment comprises two housing segments which are made from a biocompatible material that can be dissolved in the intestine/stomach, so that the two housing segments dissolve after a certain dwell time in the body of the patient. For the purpose of the endoscopic positioning in the small intestine/stomach, the two housing segments within themselves accommodate the plastic tube in a folded state, along with two annular elastic fastening means that are used to secure the plastic tube in the small intestine/stomach and are connected to one another by elastic bands.

After the successful positioning of the one housing segment, which comprises the plastic tube and the first fastening means connected thereto, distally from the pylorus and of the other housing segment, which comprises the second fastening means connected by the elastic bands to the first fastening means or to the plastic tube, proximally to the pylorus, the housing segments dissolve and reveal the plastic tube and the two fastening means. The terms “distal” and “proximal” are thereby used in relation to the esophagus, that is, “distal” refers to those positions which, in relation to the esophagus, are located farther away therefrom, and “proximal” refers to those positions which are located closer to the esophagus.

The system in a second embodiment envisages that the plastic tube and the two annular elastic fastening means are arranged in a housing or a sleeve, from which they are ejected by means of a punch-like apparatus after a corresponding positioning in the gastrointestinal tract.

In both cases, the first fastening means connected to the plastic tube thereby produces a securing in the small intestine (duodenum), that is, distally from the pylorus, and the second fastening means effects a securing in the pyloric antrum, that is, proximal to the pylorus. To secure the position of the two fastening means, and therefore also of the plastic tube, in the small intestine and in the pyloric antrum, respectively, said fastening means apply a radial pressure to the surrounding tissue so that they anchor themselves to the wall of the small intestine or to the gastric wall in the region of the pyloric antrum. Since the vagus nerve also runs through this region, said nerve is stimulated by the securing, whereby it is assumed that a feeling of satiation is generated. Both a proximal and also a distal displacement of the fastening means are impeded by the radial securing. Depending on the initial positioning, the fastening means can be braced both on the distal and also on the proximal face of the pylorus. However, the elastic bands are thereby designed such that the two fastening means can be lifted off of both the distal and also the proximal section of the pylorus.

In the course of this lifting-off of the two fastening means, in particular due to the lifting-off of the fastening means arranged in the small intestine (duodenum) from the distal section of the pylorus, it is on the one hand possible that food or chyme from the stomach moves laterally past said fastening means into the space between the plastic tube and the wall of the small intestine, and that it is absorbed there. On the other hand, depending on the distance by which the fastening means arranged in the small intestine is removed from the distal section of the pylorus, a portion of the small intestine is exposed, so that the food is also absorbed along this exposed section.

A further problem arises from the fact that the endoscopic positioning in the case of the first embodiment of the described system is dependent on the time required for the two housing segments to dissolve in the gastrointestinal tract. This involves uncertainties in particular regarding the sedation or anesthesia of the patient. It is possible to assume a window of time in which a complete opening of the plastic tube and fastening means will take place. However, this opening can also take more or less time depending on the patient's condition, so that the operating physician must constantly affirm that the housing segments have also completely dissolved in order to allow the correct positioning of the gastrointestinal implant. A premature displacement/removal of the endo scope would result in the two fastening means not being located in their intended position if the two housing segments were to dissolve ahead of or behind schedule.

Ultimately, with each embodiment in which the gastrointestinal implant is ejected out of a housing/sleeve by means of a punch-like apparatus in order to be positioned, there is the problem that, because of the punch, the two fastening means are pressed together to such an extent that they can become wedged in one another and, as a result, are no longer able to perform their task.

### OBJECT OF THE INVENTION

A first object of the present invention is therefore to propose a gastrointestinal implant that enables an improved securing in the gastrointestinal tracts.

A second object of the invention is to propose a gastrointestinal implant which effectively and reliably prevents an absorption of food/chyme in the small intestine.

A third object of the present invention is to provide an apparatus for transoral/enteral positioning, with which apparatus the process of positioning can be carried out in a safer and more precise manner.

### DESCRIPTION OF THE INVENTION

According to the invention, the first two objects are attained by a gastrointestinal implant which comprises the following:
a tube-shaped element, embodied to transport chyme through at least one section of the human intestine;
a first fastening means having a first contact surface, which fastening means is connected to the tube-shaped element and embodied to be positioned in a section of the duodenum adjacent to the pylorus;
a second fastening means having a second contact surface, which fastening means is connected to the tube-shaped element or to the first fastening means and which is embodied to be positioned in the pyloric antrum;
a connecting element which connects the first fastening means or the tube-shaped element to the second fastening means;

wherein the connecting element is embodied such that the first and second fastening means are pressed against one another with the first and second contact surface thereof, respectively, in the position of said means in the duodenum or in the pyloric antrum, respectively, with the pylorus positioned therebetween, without the fastening means being lifted off of the pylorus.

Thus, with regard to length and elasticity, the connecting element is to be sized such that, on the one hand, it is flexible enough that the pylorus can initially be positioned between the two fastening means. In addition, the connecting element must be elastically embodied and the maximum possible stretching length must in any case allow the two fastening means to be positioned in the duodenum and the pyloric antrum. However, the connecting element must at least be sized such that, for an implant positioned in the patient, pressure is constantly applied to the pylorus by the two fastening means via the respective contact surfaces thereof so that the fastening means do not lift off of the distal and/or proximal face of the pylorus, regardless of the constantly present peristalsis of the gastrointestinal tract and/or regardless of the forces that are applied by the chyme to the gastrointestinal implant, in particular the tube-shaped element, during passage through the pylorus.

According to a preferred embodiment of the invention, it is provided that the distance between the first fastening means and second fastening means in the relaxed state of the connecting element is less than the thickness of the pylorus, preferably less than 7 mm as measured in the direction of the opening embodied by the pylorus.

With this sizing according to the invention, it is in any case ensured that the two fastening means each bear with the contact surfaces thereof against the distal and/or proximal face of the pylorus under continuous pressure. Preferably, in the relaxed state the two fastening means are less than 5 mm apart from one another, in order to clamp the pylorus with adequate firmness using the two fastening means, so that a lifting-off of the fastening means from the distal and/or proximal face of the pylorus can be prevented.

This can possibly also depend on the material of the connecting element, or the geometry and thickness thereof.

Silicone has thereby proven advantageous as a material. According to another preferred embodiment of the invention, it is therefore provided that the connecting element is made of silicone. Silicone is an excellent choice on the one hand due to its adequate elasticity for the present application case, and on the other hand because of its compatibility in relation to the human body and mechanical characteristics which are stable over a wide temperature and pH range. Of course, other biocompatible materials such as PLA, PE, latex or latex substitutes, for example, are also conceivable.

According to another preferred embodiment of the invention, it is provided that the connecting element comprises multiple bands or else is embodied in a tube shape. Whereas it is easier to fold the gastrointestinal implant with the embodiment in the form of multiple bands, the embodiment of the connecting element as a tube offers the advantage of easier application and production.

According to another preferred embodiment of the invention, it is provided that the first and second fastening means respectively comprise an opening and are preferably embodied to be essentially annular, wherein annular contact surfaces also result in this case. Thus, with a corresponding sizing of the ring diameter, the clamping forces acting on the pylorus can be transferred to the best possible extent, without blocking or constricting the opening of the pylorus, wherein it should be noted that it can possibly be specifically desired to constrict the opening of the pylorus and thus artificially limit the flow of food through the pylorus. In this context, an “annular” fastening means is also understood as meaning a fastening means for which the shape deviates from the ideal ring shape but nevertheless enables the clamping of the pylorus between the contact surfaces in that said surfaces bear against the proximal and distal face of the pylorus.

According to another preferred embodiment of the invention, it is provided that the first fastening means is embodied by a first end region of the tube-shaped element. For example, the first fastening means can be formed in that the tube-shaped element is rolled up multiple times at the one end thereof, so that a kind of reinforced collar or edge forms which has an adequate thickness for forming a contact surface and being pressed against the distal face of the pylorus, in order to clamp the pylorus together with the second fastening means and the contact surface thereof. However, it is equally possible that the first end region of the tube-shaped element is embodied with a greater wall thickness than the remaining length of the element, so that the first end region forms a kind of bead that is intended to be pressed against the distal face of the pylorus.

According to another alternative embodiment of the invention, it is provided that the tube-shaped element comprises a second end region that is made of absorbable material. In this manner, it can be ensured that the tube is initially closed during the positioning in the gastrointestinal tract and for the purposes of unfolding at the second end region thereof, which is opposite from the distal face of the pylorus, and does not become passable for transporting chyme into latter regions of the small intestine until after the positioning and unfolding have occurred. For example, the absorbable section of the tube-shaped element and the non-absorbable section can be glued together, for example with a fibrin glue.

According to another preferred embodiment of the invention, it is provided that the opening provided in the second end region of the tube-shaped element is closed by a plug that is removable, preferably by means of a fluid which can be introduced into the tube. As part of the unfolding of the tube-shaped element, it can be provided that the element is unfolded in the small intestine through an introduction of a fluid (for example, CO2 or air) via the first end region of the element.

Because of the pressure which builds up in the tube-shaped element as a result, the plug in this embodiment is released by the internal pressure after the unfolding is complete and can be discharged in a natural manner. The plug can thereby be made of any desired biocompatible material, for example, a biocompatible adhesive such as starch or hard capsule gelatins. The only essential thing is that the plug initially enables the buildup of the internal pressure in the tube necessary for the unfolding and then, when the internal pressure is increased beyond that, is released from the opening so that it can be discharged naturally.

According to another embodiment of the invention, it is provided that the tube-shaped element comprises a structural weakness, for example in the form of a perforation or a thinned wall section, which separates a second end region from the remaining tube-shaped element. The structural weakness is sized such that, similar to the case of the plug, it yields in the course of the unfolding of the tube-shaped element at a sufficient amount of internal pressure, which can for example be successively increased by a fluid that can be introduced into the tube-shaped element, whereby the second end region can separate from the reaming tube-shaped element and can be discharged in a natural manner.

In another embodiment of the invention, it is provided that the tube-shaped element comprises a second end region that is embodied to be open. As will be illustrated further below, this does not impede an unfolding of said element.

According to another preferred embodiment of the invention, it is provided that the first and second fastening means, preferably the contact surfaces thereof, respectively comprise magnetically acting sections in order to assist with the pressing-together of the fastening means with the pylorus positioned therebetween. The magnetization of the respective sections can thereby be effected in various ways. One preferred embodiment envisages that small magnets can be integrated into the sections as part of the production of the fastening means. Particularly in the case where the fastening means are manufactured from silicone, the magnets can be easily integrated into the fastening means as part of the injection molding production of the fastening means such that they are distributed across the circumference or sections of the circumference. In addition to the pressing-together with the pylorus intermediately positioned, the stability of the fastening means is thus also reinforced, since the magnets on a fastening means repel each other and thus maintain a distance from one another.

According to the invention, the third object described is attained with
an apparatus for positioning a gastrointestinal implant with two essentially annular fastening means, and
a system comprising a gastrointestinal implant with two essentially annular fastening means and an apparatus for positioning the gastrointestinal implant.

It should thereby be noted that both the apparatus for positioning a gastrointestinal implant and also the system do not necessarily need to be used together with the gastrointestinal implant described above; rather, it is also conceivable to use the proposed apparatus and the proposed system with other gastrointestinal implants of the same type, with essentially annular fastening means, such as those implants described in WO2011/062882A1, for example. However, the combination of the gastrointestinal implant proposed according to the invention, composed of the plastic tube and the two essentially annular fastening means, with the likewise proposed apparatus for positioning and the proposed system, constitute a preferred embodiment/combination of the invention.

According to the invention, the positioning apparatus for positioning a gastrointestinal implant with two essentially annular fastening means comprises the following:
an outer vessel comprising a first longitudinal axis;
an inner supporting body arranged in the outer vessel and comprising a second longitudinal axis, which supporting body enables the sliding-on of the annular fastening means;
a first mounting position for the first fastening means of the gastrointestinal implant, which mounting position is arranged between the outer vessel and the inner supporting body;
a second mounting position for the second fastening means of the gastrointestinal implant, which mounting position is arranged between the outer vessel and the inner supporting body; wherein
the outer vessel can be removed from the inner supporting body, preferably axially detached in the direction of the second longitudinal axis.

The positioning apparatus initially renders it possible to slide the two fastening means onto the inner supporting body and space them apart from one another at a distance that is greater than the distance at which the two fastening means must be positioned in the gastrointestinal tract in order to continuously clamp the pylorus between them. To prevent the two fastening means from altering their position relative to one another and on the inner supporting body during the transoral insertion of the positioning apparatus via the esophagus, the outer vessel which accommodates the inner supporting body and the slid-on fastening means is provided. Between the outer vessel and the inner supporting body, mounting positions for the two fastening means are provided which enable the position-fixing of the two fastening means according to the aforementioned distance. With the outer vessel, the outer dimensions of which are chosen such that the transoral insertion of the positioning apparatus is possible through the esophagus, there occurs a compression or shape change of the fastening means, so that the fastening means can be introduced into the body.

By matching the outer and inner diameters of the outer vessel and inner supporting body with the thickness of the fastening means, it can be ensured that the compression/shape change does not occur to such an extent that the distance between the fastening means, which is specified and set outside of the body, changes in such a way that a positioning of the one fastening means in the small intestine and of the other fastening means in the pyloric antrum is no longer possible.

Through the possibility of removing the outer vessel from the inner supporting body, the fastening means can be released from their compression. The fastening means can thus relax, whereby they increase in diameter and assume their basic shape. As a result, a position-based alignment of the fastening means distally from and proximally to the pylorus is enabled, whereupon the inner supporting body can also be removed.

Various possibilities exist for the removal of the outer vessel. For example, the entire outer vessel can be composed of a biocompatible, absorbable (dissoluble) material so that the outer vessel automatically dissolves after a certain dwell time in the gastrointestinal tract. It must thereby be ensured in any case that the removal of the outer vessel at least does not occur until the positioning apparatus protrudes through the pylorus and the mounting position of the one fastening means is in the small intestine (duodenum) and the mounting position of the other fastening means is in the pyloric antrum. After the fastening means have assumed their basic shape, they can already be braced with their contact surfaces on the proximal and distal face of the pylorus, so that the inner supporting body can also be subsequently removed.

The mounting positions themselves can be embodied in various ways. The embodiment of the mounting positions has an influence on the manner of the position-fixing for the fastening means.

In a preferred embodiment, the mounting positions result from a simple clamping of the fastening means between the outer vessel and the inner supporting body. It is thereby provided that the first and second mounting positions respectively comprise an outer limit constituted by the outer vessel and an inner limit constituted by the inner supporting body. The distance between the limits is thereby selected such that the two fastening means are position-fixed, that is, clamped between the outer vessel and the inner supporting body. In other words, it renders it possible for the positioning apparatus according to the invention, through a corresponding sizing of the outer vessel and inner supporting body, to clamp the fastening means therebetween, whereby the fastening means, as described, are compressed or change their shape and can be inserted into the gastrointestinal tract transorally.

To enable an optimization of the mounting positions with a view to the position-fixing, is provided according to a preferred embodiment of the invention that the inner supporting body comprises two grooves running along the surface thereof for at least partially accommodating the first and second fastening means, wherein the minimum distance of the grooves to one another is preferably between 3 mm and 70 mm, particularly preferably between 5 mm and 30 mm.

The grooves can run along the surface of the inner vessel in any desired manner and must be matched to the fastening means, in particular to the sizing thereof and the necessary compression and position thereof. Thus, according to a preferred embodiment, it can be provided that the grooves run on planes that are perpendicular to the longitudinal axis of the inner supporting body in a top view and/or side view. According to another embodiment of the invention, however, it can also be provided that the grooves run on planes that are oblique to the longitudinal axis of the inner supporting body in a top view and/or side view. It is also conceivable that one of the grooves runs on a plane that is perpendicular to the longitudinal axis of the inner supporting body in a top view and/or side view, while the other groove runs on a plane that is oblique to the longitudinal axis of the inner supporting body in a top view and/or side view.

Preferably, the outer vessel is thereby embodied in a sleeve shape and—in the event that the outer vessel is not embodied to be fully absorbable, as has been described further above—it can be alternatively provided that the distal end region of the outer vessel is embodied to be closed and removable or else closed and openable.

Whereas the removability of the distal end region can be very easily achieved in that said end region is made from a biocompatible, absorbable (dissoluble) material, another alternative embodiment of the invention envisages that the outer vessel comprises a structural weakness, preferably in the form of a predetermined breaking point, which allows the removal of the distal end region from the remaining outer vessel and thus the openability through an application of pressure to the end region. A structural weakness of this type can, for example, be achieved in that the distal end region is connected to the rest of the outer vessel via a perforated section or a section having a thinner wall cross section. It is thus ensured that the axial pulling-off of the outer vessel from the inner supporting body can take place in a direction that is opposite of the insertion direction for the positioning apparatus. The separated distal end can be discharged from the body naturally.

In a preferred embodiment of the invention, it is provided that the structural weakness does not run across the entire circumference of the outer vessel, but rather only across a section of the circumference and enables a partial detachment of the distal end region. The distal end region partially detached in this manner thus remains connected to the outer vessel and can be removed from the body again together with the outer vessel against the insertion direction.

In another embodiment of the invention, it can also be provided that the distal end region of the outer vessel is glued to the remaining outer vessel and the structural weakness occurs by adjusting the strength of the adhesive.

To allow the gastrointestinal implant to achieve the intended effect, it must be ensured that the tube-shaped element can unfold with its entire length in the intestine, preferably before the gastrointestinal implant is secured by the two fastening means, and that it does not comprise any sections which block the passage of the chyme. The longer the tube-shaped element is, the more difficult a complete unfolding becomes. Extensive tests have shown that the provision of an aligning section, which preferably comprises a cup-shaped holding volume, facilitates or enables the necessary complete unfolding. The aligning section can thereby constitute the distal end of the inner supporting body, so that the gastrointestinal implant with its tube-shaped element and two fastening means can be slid onto the inner supporting body starting at this end region before the transoral insertion of the positioning apparatus, until the two fastening means have reached their intended mounting positions. At this point in time, the tube-shaped element envelops the aligning section. Because the aligning section has a substantially smaller length than the overall length of the tube-shaped element, the element would protrude past the aligning section by a considerable residual length. However, the preferably cup-shaped holding volume renders it possible to accommodate this residual length in the holding volume.

To allow the unfolding of the plastic tube, it can be provided that the inner supporting body comprises a channel running through the body, which channel is provided with an outlet opening in the aligning section. Via this channel, a filler medium, preferably CO₂ or air, can be applied to the tube-shaped element, whereby the channel unfolds into the small intestine from the holding volume. The application of the filler medium can thereby occur in a pulsed manner or by means of a constant pressure. Preferably, the application takes place at a pressure of 0.1 to 9 bar, particularly preferably at no more than 4 bar.

According to a particularly preferred embodiment of the invention, it is provided that the gastrointestinal implant according to the invention and the apparatus according to the invention for positioning a gastrointestinal implant constitute a system, that is, are used together. Such a system comprises:

an apparatus which comprises the following:
an outer vessel comprising a first longitudinal axis;
an inner supporting body arranged in the outer vessel and comprising a second longitudinal axis;
a first mounting position for the first fastening means, which mounting position is arranged between the outer vessel and the inner supporting body;
a second mounting position for the second fastening means, which mounting position is arranged between the outer vessel and the inner supporting body; wherein
the outer vessel can be removed from the inner supporting body, preferably axially detached in the direction of the second longitudinal axis;

and a gastrointestinal implant, which comprises the following:
a tube-shaped element, embodied to transport chyme through at least one section of the human intestine;
an first fastening means connected to the tube-shaped element and comprising an opening, embodied to be positioned in a section of the duodenum adjacent to the pylorus;
a second fastening means connected to the tube-shaped element or to the first fastening means and comprising an opening, embodied to be positioned in the pyloric antrum;
a connecting element which connects the first fastening means or the tube-shaped element to the second fastening means;

and wherein the first and second fastening means are slid onto the inner supporting body and, position-fixed in the first and second mounting position, respectively, are compressed to a first size and/or assume a first position, and when the outer vessel is removed expand to a second size and/or assume a position different from the first position.

The system according to the invention initially allows the operating physician to precisely position the gastrointestinal implant in the gastrointestinal tract of the patient with the aid of the positioning apparatus such that the one mounting position is located in the small intestine, or more accurately in the duodenum, and the other mounting position is located in the pyloric antrum. For this purpose, it can be provided that markings are affixed to the outer vessel which indicate to the operating physician how deeply the first and second mounting positions have already been inserted into the body of the patient, from which the location of the mounting positions for the fastening means in relation to the pylorus can then be concluded.

If the operating physician concludes that the first and second mounting positions are correctly positioned, the physician can remove the outer vessel after the tube has been unfolded, either by axially pulling the outer vessel off or else by waiting for an amount of time until the outer vessel has been absorbed. After the outer vessel has been removed, the fastening means can expand or assume a securing position, whereby the securing of the gastrointestinal implant in the gastrointestinal tract is complete and the positioning apparatus can once again be removed.

Preferred embodiments of the system are described in the dependent claims.

### BRIEF DESCRIPTION OF THE DRAWINGS

The present invention will now be described in greater detail with the aid of one or more figures illustrating exemplary embodiments. In this matter:

FIG. 1 shows a gastrointestinal implant comprising a tube-shaped element and two fastening means

FIG. 2 shows a gastrointestinal implant arranged in the human gastrointestinal tract

FIG. 3 shows an embodiment of a fastening means of a gastrointestinal implant with magnetic sections

FIG. 4 shows an embodiment of a fastening means with a supporting element that has a variable length

FIG. 5 shows an embodiment of a fastening means with coil spring elements as supporting elements

FIG. 6 shows an embodiment of a fastening means with a coil spring element as supporting elements

FIG. 7 shows an embodiment of a fastening means

FIG. 8 shows an embodiment of a fastening means

FIG. 9 shows a first embodiment of an apparatus for positioning a gastrointestinal implant in the human gastrointestinal tract before the removal of the outer vessel

FIG. 10 shows a second embodiment of an apparatus for positioning a gastrointestinal implant in the human gastrointestinal tract before the removal of the outer vessel

FIG. 11 shows different possible embodiments of grooves on the inner supporting body

FIG. 12 shows the first embodiment of the apparatus for positioning a gastrointestinal implant in the human gastrointestinal tract with an applied gastrointestinal implant

FIG. 13 shows the second embodiment of the apparatus for positioning a gastrointestinal implant in the human gastrointestinal tract with an applied gastrointestinal implant

FIG. 14 shows a detailed view of a section of the inner supporting body with oblique grooves

FIG. 15 shows another detailed view of a section of the inner supporting body with straight grooves

FIG. 16 shows another detailed view of a section of the inner supporting body with recesses

FIG. 17 shows a positioning apparatus with an applied gastrointestinal implant shortly after the transoral insertion

FIG. 18 shows a positioning apparatus with an applied gastrointestinal implant with a pulled-back outer vessel

FIG. 19 shows a positioning apparatus with an applied gastrointestinal implant with a detached distal end region of the tube-shaped element

FIG. 20 shows a positioning apparatus with an applied gastrointestinal implant with an expanded first fastening means

FIG. 21 shows a positioning apparatus with an applied gastrointestinal implant with an expanded second fastening means

FIG. 22 shows an embodiment of an outer vessel with a limit stop

FIG. 23 shows an embodiment for an inner supporting body

FIG. 24 shows an embodiment for an inner supporting body

### WAYS OF EMBODYING THE INVENTION

### Gastrointestinal Implant

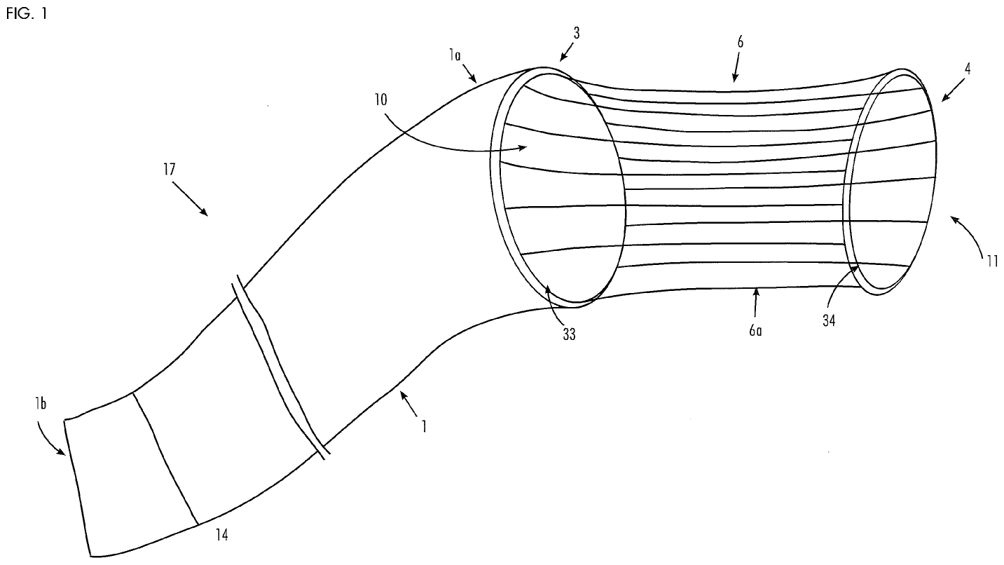

FIG. 1 shows a gastrointestinal implant 17 according to the invention, comprising a tube-shaped element 1 and two fastening means 3 and 4 that are connected to one another via a connecting element 6 and are used to position the tube-shaped element 1 in the intestine. It has the task of transporting the chyme entering the intestine via the stomach through at least one section of the human intestine without the chyme being digested. Preferably, the tube-shaped element 1 is, at least in sections, made of silicone or fluorosilicone or another biocompatible material that is capable of dwelling in the stomach or intestine for a longer period without decomposing and which exhibits adequate flexibility to be transported into the gastrointestinal tract via the mouth and esophagus, preferably in a folded/invaginated state.

The length of the tube-shaped element 1 can vary depending on the purpose of use. The longer the tube-shaped element 1 is embodied to be, the longer the region of the intestine through which the chyme can be transported without absorption, and therefore the larger and more quickly attained the weight reduction that is to be achieved. The wall thickness of the tube-shaped element 1 can also vary in sections, in order to possibly increase the resistance to the natural peristalsis movements of the gastrointestinal tract.

FIG. 1 shows the tube-shaped element 1 in an unfolded state. The one (distal) end 1b thereof is therefore—initially—closed, and at the other (proximal) end 1a thereof, the tube-shaped element is tightly connected to or fabricated in a single piece with the first fastening means 3, so that no chyme can pass between the fastening means 3 and the tube shaped element 1. As will be described in greater detail below, the closed end 1b is initially used to unfold the tube-shaped element 1 to its intended length in the intestine.

The position-fixing of the tube-shaped element 1 in the intestine occurs via the fastening means 3 and 4, or the contact surfaces 33 (fastening means 3) and 34 (fastening means 4), which are used to clamp the pylorus 7 therebetween and thus to enable an anchoring of the gastrointestinal implant in the gastrointestinal tract. For the clamping function to be performed, it is provided that the two fastening means 3 and 4 are connected to one another via a connecting element 6. The connecting element 6 is embodied to be elastic and is made of, and is preferably composed of a number of bands 6a, or alternatively of a tube-shaped connecting element (not illustrated). Both bands 6a and also the tube-shaped connecting element are embodied to be guided through the opening of the pylorus. The elasticity of the connecting element 6 is thereby chosen such that, if a distance between the two contact surfaces 33 and 34 is exceeded, which distance is slightly smaller than the typical thickness of the pylorus, a pulling force acts on the two fastening means 3, 4 and therefore on the contact surfaces 33 and 34, which force—regardless of the gastrointestinal tract movements caused by peristalsis and regardless of the closing and opening movements of the pylorus—prevents the detachment of the respective contact surface 33, 34 from the distal and/or proximal face of the pylorus, so that the contact surfaces 33, 34 bear against the faces 31, 32 of the pylorus with sustained pressure.

Preferably, the fastening means 3, 4 are also embodied to be adequately elastic so that an adaptation to the different dimensions of the gastrointestinal tract, and to the previously mentioned movements due to peristalsis and the closing and opening movements of the pylorus, is possible. It is thereby provided that the two fastening means 3, 4 have in a relaxed state a roughly annular shape, so that on the one hand adequate contact surface is available to clamp the pylorus on both sides, but so that on the other hand the fastening means 3, 4 also have an adequately sized flow opening 10, 11 to allow unimpeded passage of the chyme into the small intestine, specifically the duodenum. It should be noted, however, that larger temporary deviations from the circular shape do not significantly impair the functionality of the fastening means 3, 4. Preferably, the fastening means 3, 4 are embodied to be circular.

In alternative embodiment of the invention, it can be provided that the inner diameter of the approximately annular fastening means 3, 4, which diameter defines the flow opening 10, 11, is not greater than 5 mm, so that passage of the chyme from the stomach into the intestinal tract is delayed to allow the more rapid onset of a feeling of satiation.

Preferably, the two fastening means 3, 4 and also the connecting element 6 are made of the same material as the tube-shaped element 1. However, due to the requirements according to the invention for the connecting element 6, it is also conceivable that said element is composed of a material different from the fastening means 3, 4.

In principle, however, all materials that are adequately elastic and biocompatible are conceivable for embodying the fastening means 3, 4 and/or the connecting element 6.

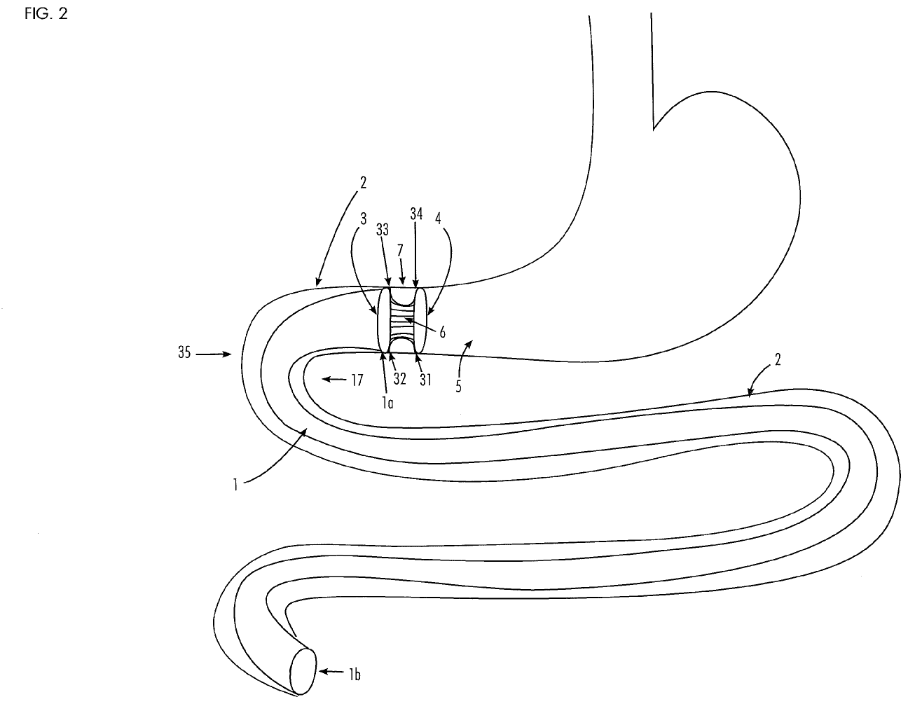

FIG. 2 shows the gastrointestinal implant arranged in its final position in the gastrointestinal tract. The tube-shaped element 1 is thereby already located, in a completely unfolded manner, in the intestine 35, starting with its proximal end 1a in the duodenum 2. The two fastening means 3, 4 are arranged distally from and proximally to the pylorus 7, respectively. The connecting element 6, which is composed of multiple bands 6a, is embodied such that it pulls the two fastening means 3, 4 to one another in the illustrated final position of the gastrointestinal implant 17, so that the contact surfaces 33, 34 of said means continuously bear against the proximal 31 and distal 32 faces of the pylorus 7, respectively, and permanently clamp it between them on both sides, without the contact surfaces 33, 34 lifting off of the proximal 31 and/or 32 distal face of the pylorus 7.

In this final position, the distal end 1b of the tube-shaped element 1 is already open, so that chyme can fully pass through the tube-shaped element 1.

The two fastening means 3, 4 can comprise magnetically acting sections 29, 30 which act in such a way that they support the pulling force of the connecting element 6 and therefore the clamping effect. Furthermore, the magnetic sections 29, 30 facilitate the position-fixing of the fastening means 3,4 such that the flow openings 10, 11, regardless of the peristalsis and opening and closing movements of the pylorus 7 taking place, essentially remain in constant concentric alignment, or in the case of deformed, non-circular flow openings, remain aligned such that the passage of the chyme is not impeded or even blocked. The radial shifting, even temporarily, of the fastening means 3, 4 can thereby be prevented.

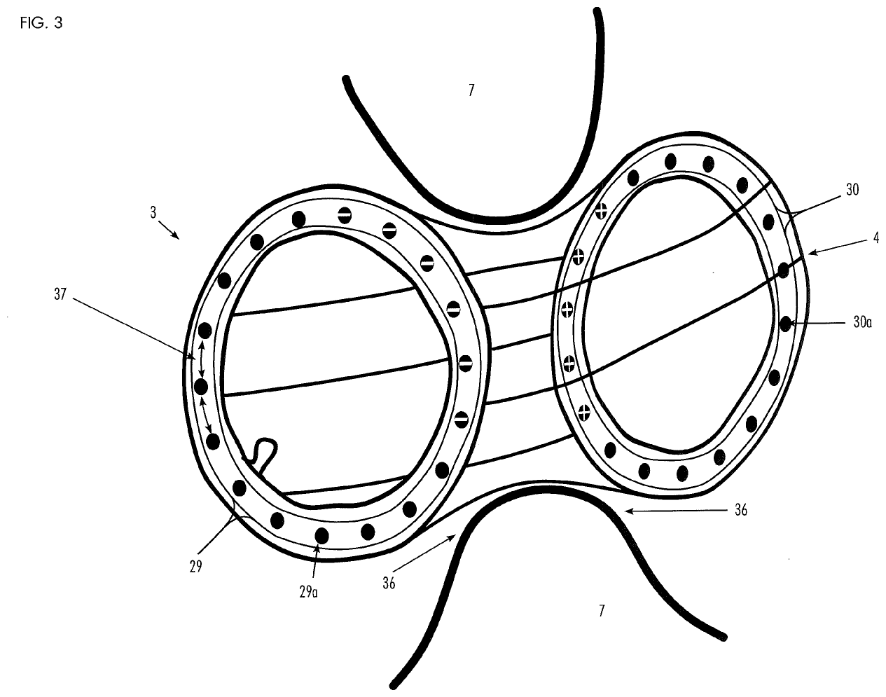

FIG. 3 shows fastening means 3, 4 having magnetic sections 29, 30, wherein in this exemplary embodiment, each of the fastening means 3, 4 respectively comprises a magnetic section 29, 30 that spans the entire circumference of the fastening means 3, 4. However, it is also conceivable that, on each fastening means 3, 4, multiple magnetic sections are provided which are then accordingly sized to be smaller and, for example, are separated from one another by non-magnetically acting sections.

In the exemplary embodiment illustrated, each section 29, 30 comprises multiple individual magnets 29a, 30a which are arranged such that the individual magnets 29a of the first fastening means 3 and the individual magnets 30a of the second fastening means 4 attract one another and thus support the clamping effect (see arrow with reference numeral 36).

At the same time, the illustrated arrangement renders it possible that the individual magnets 29a and 30a repel one another (see arrow with reference numeral 37). In this manner, it is possible to support the specified shape, preferably the specified ring shape, and counteract shape changes that would arise as a result of the compression of the fastening means 3, 4 (for example, due to peristalsis), such as undesired torsion.

Notwithstanding the above, the surface of the fastening means 3, 4 can also be roughened in order to increase the torsion resistance.

Because the pylorus 7 is a sphincter, it is, as mentioned above, advantageous if the two fastening means 3, 4 also assume an approximate ring shape in their final position in the gastrointestinal tract, in which position the gastrointestinal implant 17 is secured, so that the contact surfaces 33, 34 of said means can bear to the best possible extent against the proximal and distal faces 31, 32 of the pylorus 7, respectively. It should thereby be kept in mind that, for the purposes of transoral insertion of the gastrointestinal implant 17 via the esophagus, the fastening means 3, 4 must initially be compressed, at least in sections, which is normally accompanied by a shape change. The fundamentally elastic embodiment of the fastening means 3, 4 allows on the one hand the required compression and shape change thereof, but on the other hand also the expansion thereof into a final state in which the clamping of the pylorus is to occur.

In a first embodiment, it is thereby provided that the fastening means 3, 4 are respectively composed of an elastic ring of biocompatible material, such as silicone or fluorosilicone or the like, for example, which satisfies the elasticity requirements described above.

In another alternative embodiment it is provided that the elastic ring of one or both fastening means 3, 4 is made of biocompatible material such as silicone, fluorosilicone or the like, for example, and comprises an interior volume in which a supporting element 41, for example made of polyethylene (PE), is arranged.

The supporting element 41 has the task of shaping the fastening means 3, 4 such that said means has greater dimensional stability in its intended final position, and to thus optimize the clamping effect.

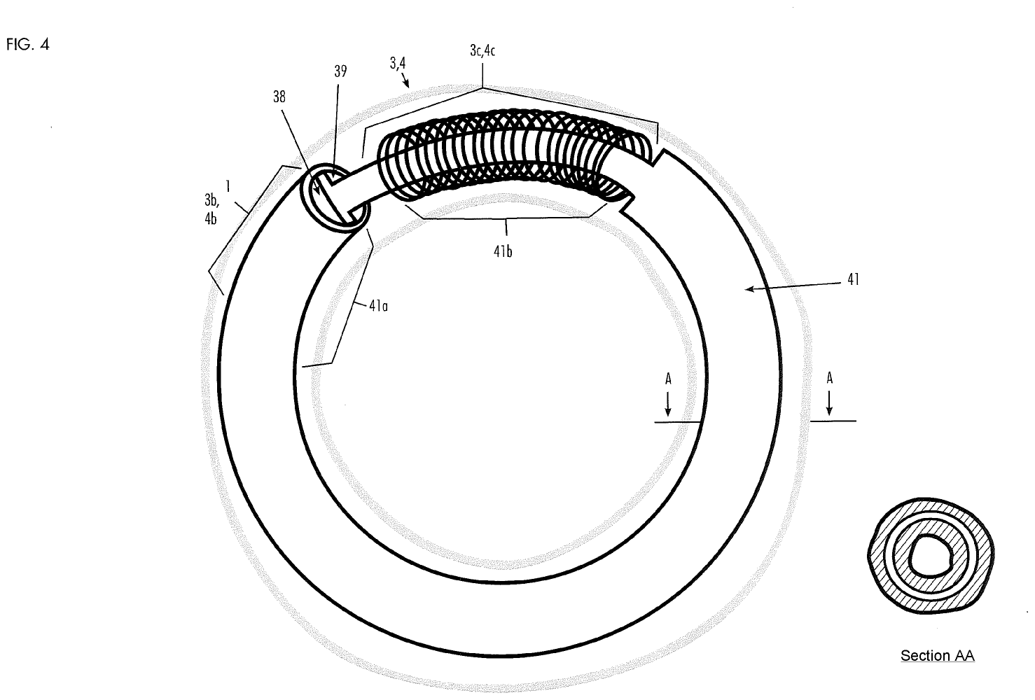

To nevertheless allow an adequate compression during the transoral insertion of the gastrointestinal implant, it can be provided according to this alternative embodiment that a supporting element 41 of this type is embodied in a divided manner and comprises a first end section 41a with an interior volume 38 in which the second end section 41b resulting from the division is displaceably held. The second end section 41b has for this purpose a smaller cross section than the remaining supporting element 41, so that it can easily be inserted into the first end section 41a comprising an interior volume 38 for this purpose, as can be seen in FIG. 4.

In this manner, the fastening means 3, 4 are easier to compress and can therefore be better adapted to peristalsis and the opening and closing movement of the pylorus 7.

To optimize the clamping capacity of a fastening means 3, 4 in this embodiment, it can be provided that the end section 41b is surrounded by a coil spring element 40 which constantly pulls the end section 41b as far as possible out of the interior volume 38, in order to constantly keep the circumference of the annular fastening means 3, 4 as large as possible, and to thus make available the largest possible contact surface 33, 34 for clamping the faces 31, 32 of the pylorus 7.

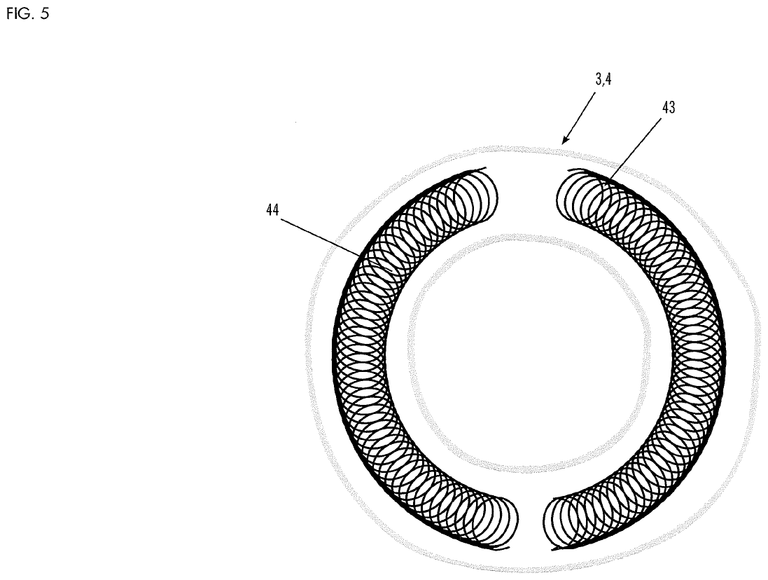

FIG. 5 shows an embodiment of a fastening means 3, 4 similar to the embodiment illustrated in FIG. 4, with the difference that, instead of the divided supporting element 41 with telescoping end sections 41a and 41b, it comprises two coil spring elements 43, 44 that function as supporting elements.

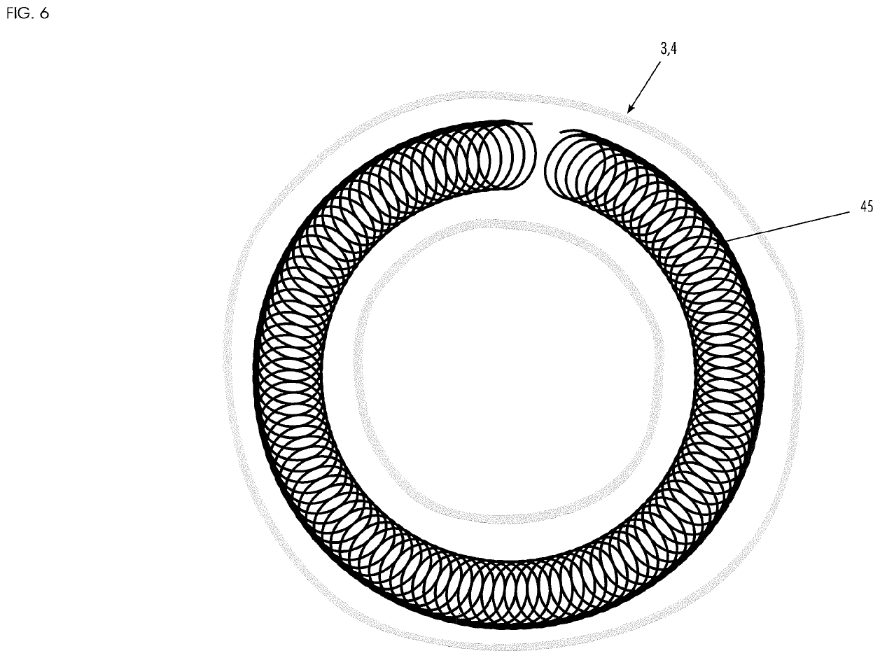

Alternatively, it is also possible to provide only a single coil spring element 45 that is embodied in a divided manner corresponding to the supporting element 41 of the embodiment according to FIG. 4, as is illustrated in FIG. 6.

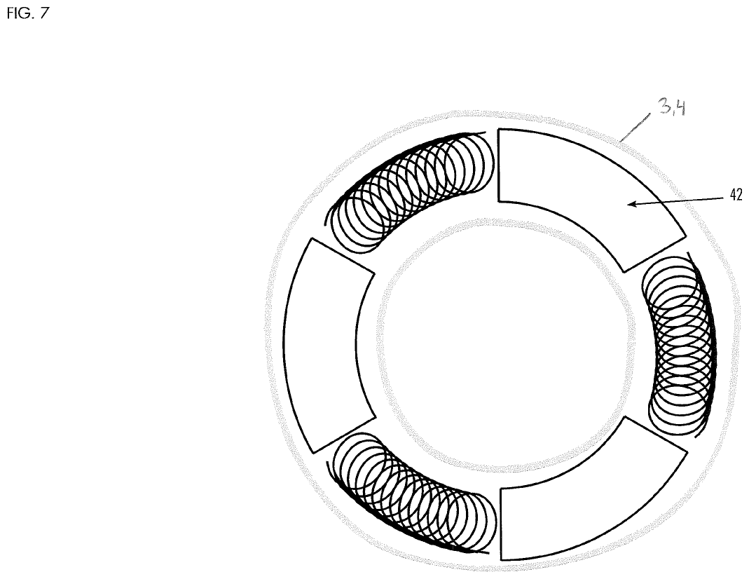

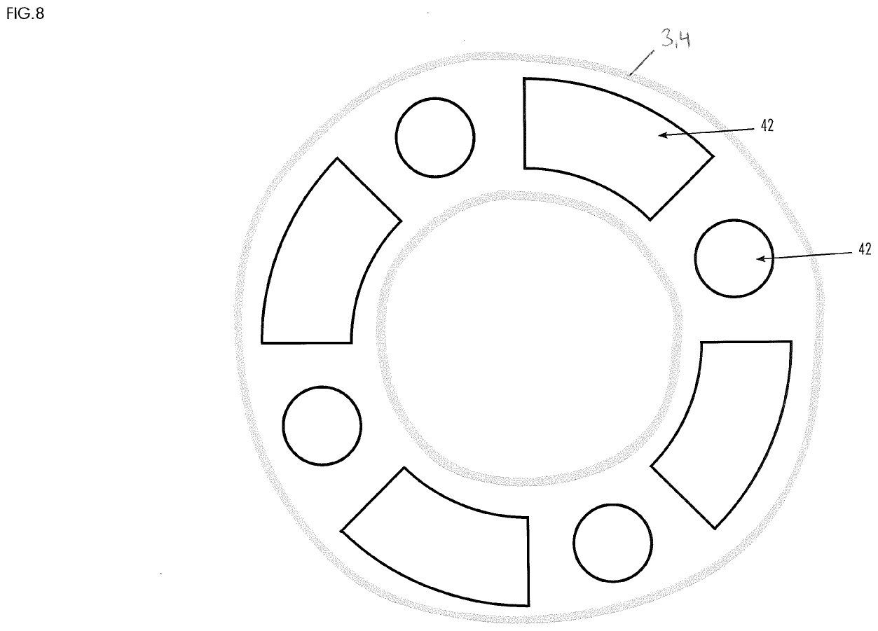

FIG. 7 and FIG. 8 each show alternative embodiments of a fastening means 3, 4 with additional supporting elements 42, for example, made of PE (polyethylene).

### Positioning Apparatus for Positioning a Gastrointestinal Implant in the Gastrointestinal Tract and System Comprising a Gastrointestinal Implant and a Positioning Apparatus

In order to transport the gastrointestinal implant as described in FIGS. 1 through 8 into its final position in the gastrointestinal tract, a positioning apparatus 18 is provided. In principle, the positioning apparatus 18 described below can also be used for the positioning of other gastrointestinal implants which differ from those depicted in FIGS. 1 through 8, provided that these other gastrointestinal implants comprise two essentially annular fastening means that are intended to bear against the distal and proximal faces of the pylorus, in particular also for gastrointestinal implants in which the connecting element 6 is not embodied according to the invention and therefore allows a lifting-off of the contact surfaces 33, 34 from the distal and/or proximal face 31, 32 of the pylorus 7.

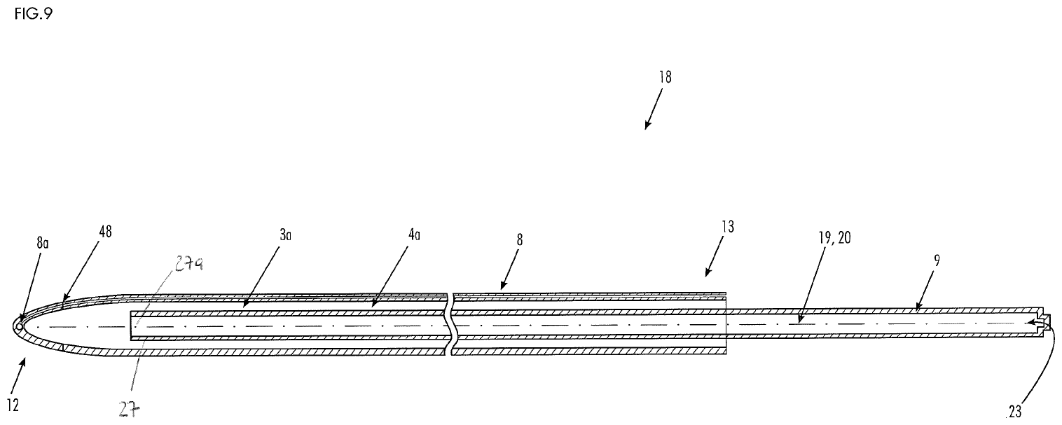

FIG. 9 shows a positioning apparatus according to the invention, composed of an outer vessel 8 with an interior volume in which an inner supporting body 9 is arranged. The outer vessel 8 has the option 8a for fastening a guide wire, the purpose of which will be explained further below.

The distal end region 12 of the outer vessel 8, that is, the end region which is arranged in the frontal position in the course of the transoral insertion into the esophagus, is for the purposes of insertion embodied to be rounded, formed in a cartridge shape and closed, but removable or at least openable. The removability can be achieved in that the end region 12 is made of a biocompatible, absorbable (dissoluble) material. The openability is rendered possible in that the outer vessel can comprise a structural weakness, preferably in the form of a predetermined breaking point, which allows the removal of the distal end region 12 from the remaining outer vessel through an application of pressure to the end region 12 from the inside. A structural weakness of this type can, for example, be achieved in that the distal end region 12 is connected to the rest of the outer vessel 8 via a perforated section or a section having a thinner wall cross section. In a preferred embodiment of the invention, it is provided that the structural weakness does not run across the entire circumference of the outer vessel 8, but rather only across a section of the circumference and enables a partial detachment of the distal end region.

The proximal end region 13 is used to introduce the inner supporting body 9 and, in a preferred embodiment, comprises limit stops (not shown) in order to place the outer vessel 8 and inner supporting body 9 in a defined position relative to one another.

The inner supporting body 9 preferably comprises a circular cross section in order to allow the sliding-on of the fastening means 3, 4.

The distance between the outer vessel 8 and inner supporting body 9 is selected such that the fastening means 3, 4 can be clamped between the two components and thus position-fixed. In this manner, a first mounting position 3a for the first fastening means 3 and a second mounting position 4a for the second fastening means 4 are formed. These are shown purely by way of example in FIG. 9.

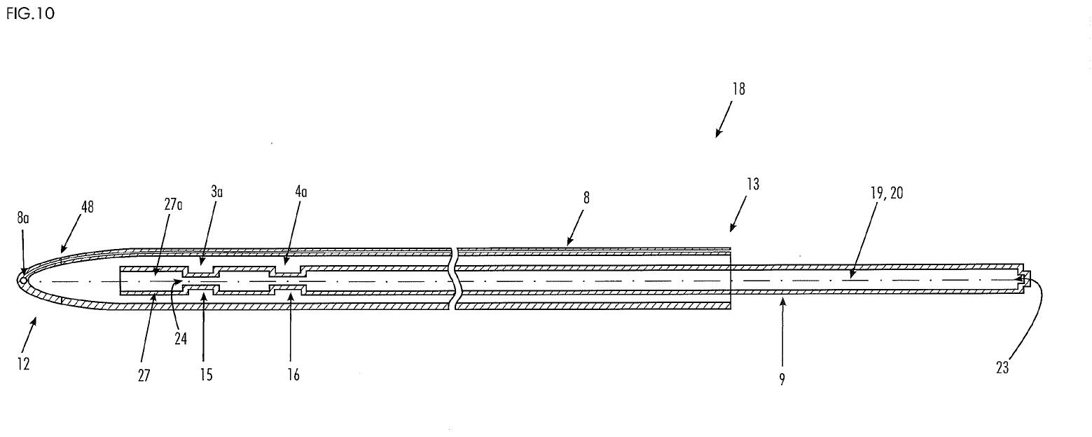

In an alternative embodiment as shown in FIG. 10, the first and second mounting positions 3a, 4a are formed by grooves 15, 16 on the surface of the inner supporting body 9, in which the two fastening means 3, 4 can be at least partially accommodated, that is, the fastening means 3, 4 can either be completely arranged in the grooves 15, 16 or only partially arranged in said grooves and protrude past them. The distance of the grooves 15, 16 from one another is preferably between 3 mm and 70 mm, in particular preferably between 5 mm and 30 mm. The embodiment of the mounting positions 3a, 4a as grooves has the advantage that the position-fixing does not occur in a frictional engagement, but rather by means of a positive fit, whereby an inadvertent shifting of the fastening means 3, 4 can in principle be eliminated and the exact distance between the fastening means 3, 4 on the inner supporting body 9 is also easier to set.

The course of the grooves 15, 16 on the surface of the inner supporting body 9 must be matched to the embodiment and/or sizing and/or elasticity of the fastening means 3, 4 and to the required/desired degree of compression.

Thus, according to a preferred embodiment of the invention, it can be provided that the grooves 15, 16 run on planes that are arranged perpendicularly to the longitudinal axis 20 of the inner supporting body 9 in a top view and/or side view. According to another embodiment of the invention, however, it can also be provided that the grooves 15, 16 run on planes that are arranged obliquely to the longitudinal axis 20 of the inner supporting body 9 in a top view and/or side view. It is also conceivable that one of the grooves runs on a plane that is perpendicular to the longitudinal axis 20 of the inner supporting body 9 in a top view and/or side view, while the other groove runs on a plane that is oblique to the longitudinal axis 20 of the inner supporting body 9 in a top view and/or side view. The oblique positioning of the grooves 15, 16 renders possible a lesser degree of compression of the fastening means 3, 4.

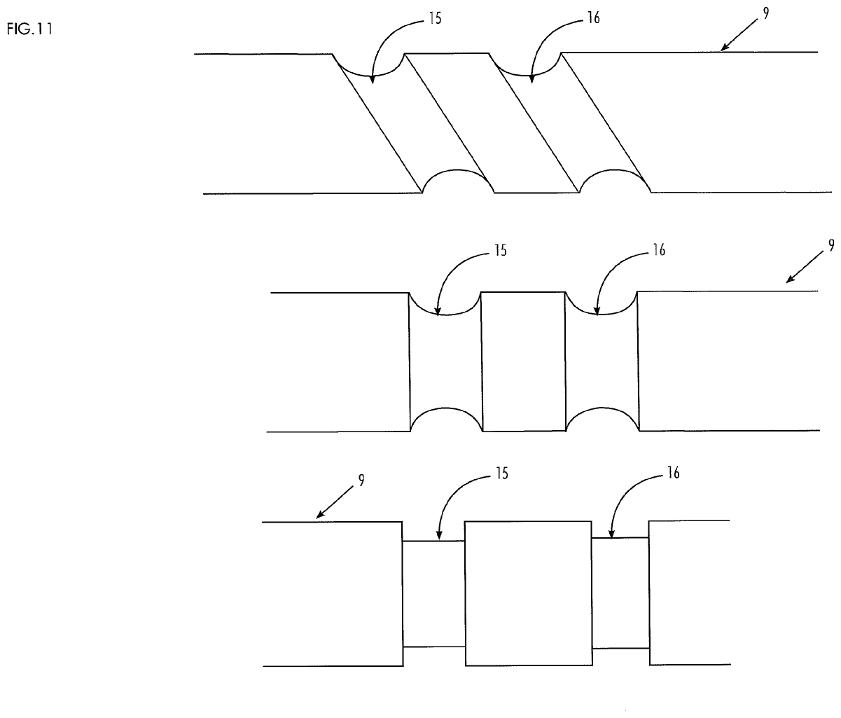

FIG. 11 shows, by way of example, different ways of embodying the grooves 15, 16 on the inner supporting body 9.

The mounting positions 3a, 4a can also be formed by a combination of the embodiments described above, so that the fastening means 3, 4 accommodated sectionwise in the grooves 15, 16 can be simultaneously clamped between the inner supporting body 9 and the outer vessel 8.

Irrespective of the specific embodiment of the mounting positions, the distance between the mounting positions 3a, 4a is always chosen such that it is embodied to be slightly larger than the thickness of the pylorus 7 and such that, when the two fastening means 3, 4 are located at the mounting positions 3a, 4a, the connecting element 6 is already tensioned and a pulling force is exerted on each of the two fastening means 3, 4.

In reference to FIGS. 9 and 10, it can be provided according to the embodiments illustrated therein that the inner supporting body 9, at the distal end region 22 thereof, comprises an aligning section 27 for the tube-shaped element 1, as is specifically illustrated in FIG. 10. The aligning section 27 is used to initially allow the tube-shaped element 1 to be accommodated in a compact, space-saving state in order to enable or facilitate the transoral insertion of the gastrointestinal implant. However, the aligning section 27 is also subsequently used to enable the complete unfolding of the tube-shaped element 1 out of the compact state.

For this purpose, the aligning section 27 can, according to a preferred embodiment of the invention, be provided with a preferably cup-shaped holding volume 27a, as is illustrated in FIGS. 9 and 10, wherein the open end of the holding volume 27a points in the direction of the distal end of the outer vessel 8.

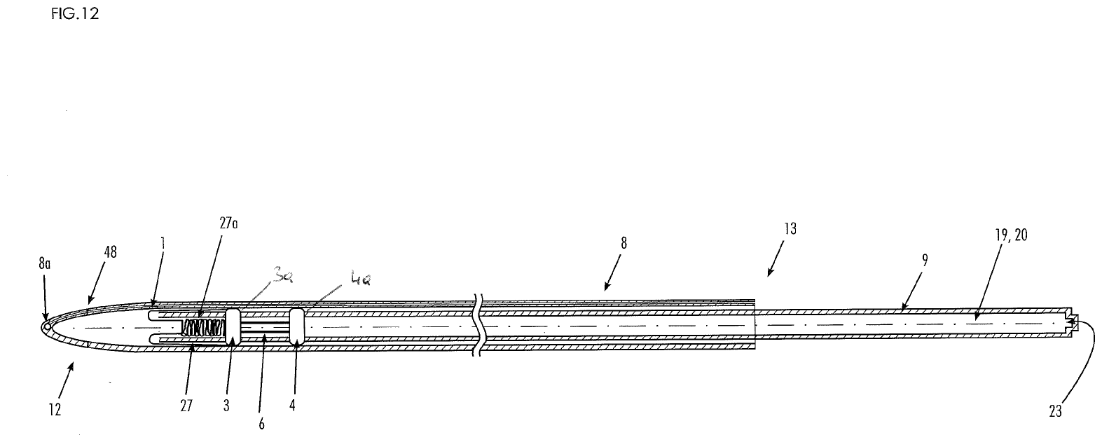

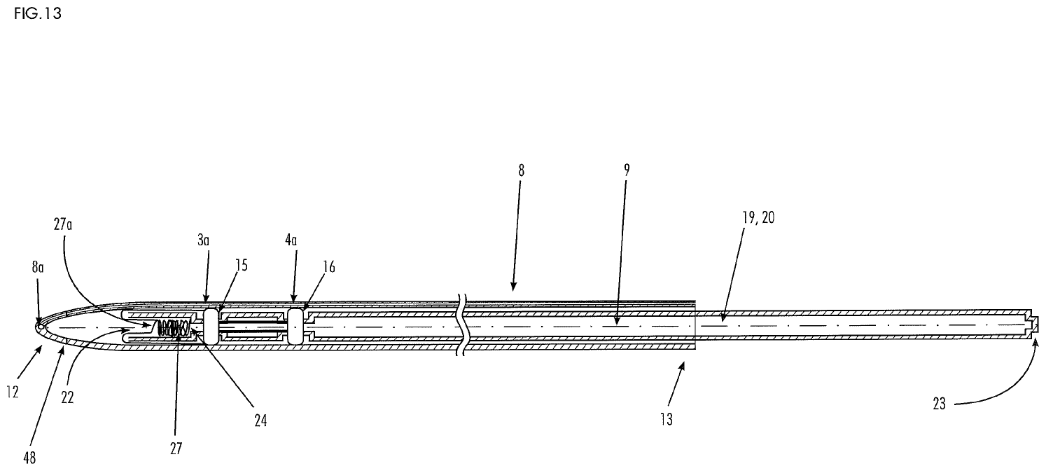

The application of a gastrointestinal implant on the positioning apparatus 18 is shown in FIGS. 12 and 13, wherein FIG. 12 shows the application on a positioning apparatus 18 as illustrated in FIG. 9, and FIG. 13 shows the application on a positioning apparatus 18 as illustrated in FIG. 10.

The gastrointestinal implant 1, as illustrated for example in FIGS. 1 through 8, is thereby initially slid onto the inner supporting body 9. In the event that the inner supporting body 8 is provided with grooves 15, 16, the fastening means 3, 4 are mounted with at least a portion of their cross section in these grooves 15, 16, which form the mounting positions 3a, 4a. Regardless of whether the mounting positions 3a, 4a are formed by grooves 15, 16 or else solely by the distance between the outer vessel 8 and the inner supporting body 9 as is illustrated in FIG. 12, the fastening means 3, 4 are in this state compressed, since the outer vessel 8 prevents the expansion thereof to a larger diameter.

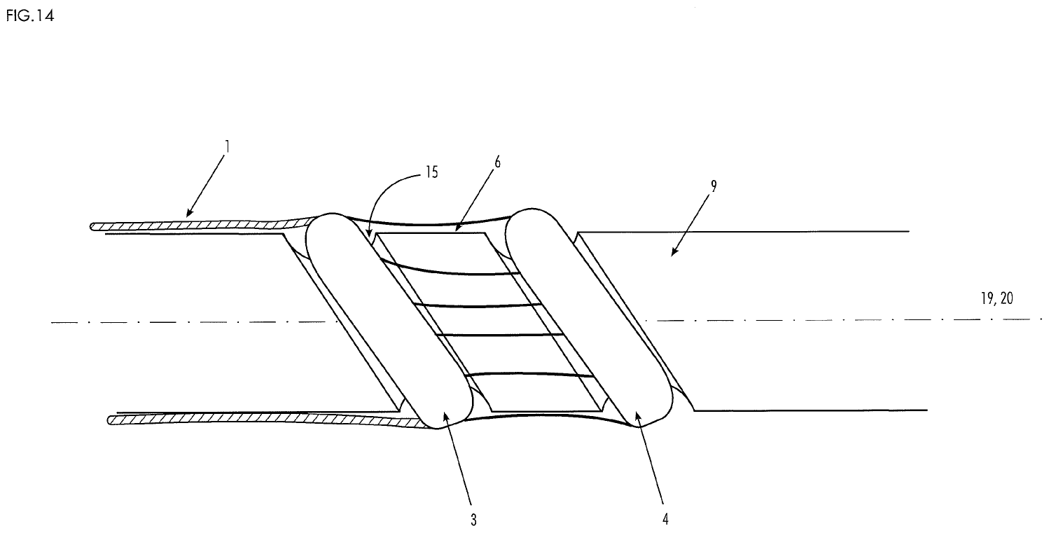

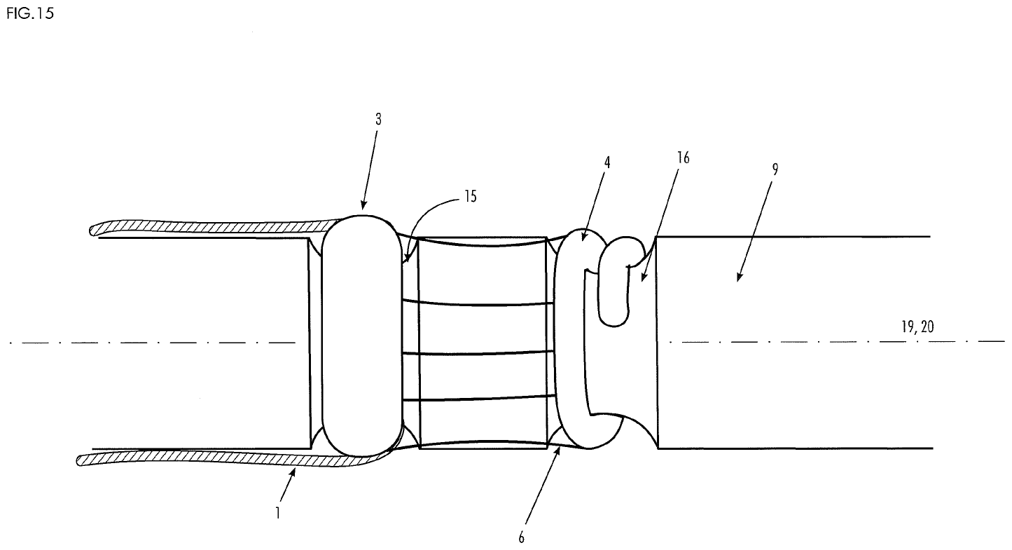

FIGS. 14 and 15 show, purely by way of example, the region of the grooves 15, 16 in an enlarged illustration, wherein the grooves 15, 16 in FIG. 14 are embodied obliquely to the longitudinal axis 20 and in FIG. 15 are embodied to be straight. In FIG. 15, also purely by way of example, the one fastening means 4 is embodied helically, in order to demonstrate that the fastening means 3, 4 can in principle also have shapes different from those previously illustrated. Whereas fastening means 3, 4 in the illustrated embodiments have a circular cross section, other cross section geometries are also conceivable, for example, rectangular cross sections. In this case, it is also conceivable to embody the grooves 15, 16 with a rectangular cross section.

With the sliding of the implant 17 onto the inner supporting body 9, the tube-shaped element 1 is automatically pulled over the aligning section 27, so that it envelops said section. That section of the tube-shaped element 1 which protrudes past the aligning section 27, that is, which can no longer be slid on, is initially invaginated. This invaginated section can then be either left in front of the aligning section 27 or stuffed into the holding volume 27a and therein accommodated preferably in a folded or differently compressed manner.

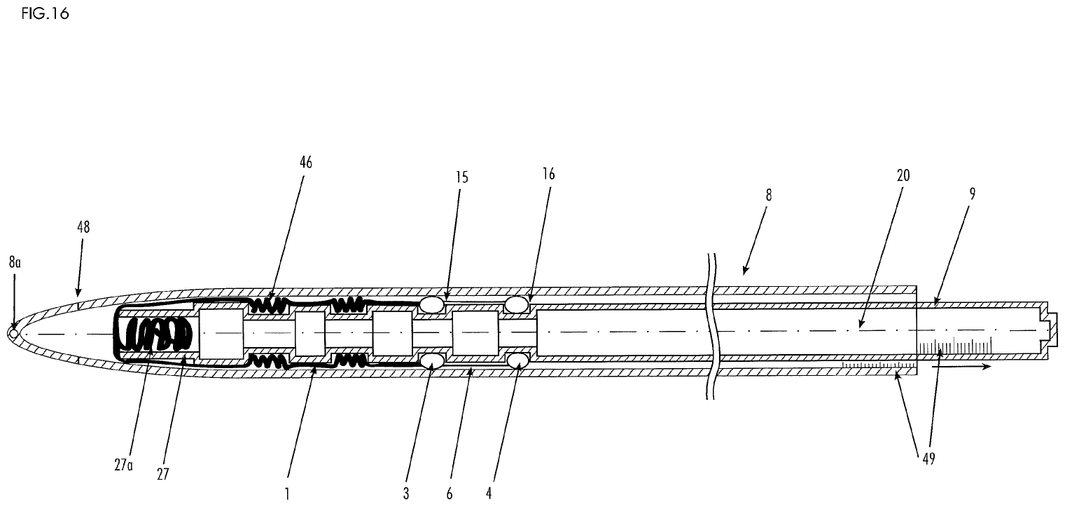

Additionally, it can be provided to envisage recesses 46 on the surface of the inner supporting body 9, in which recesses 46 sections of the tube-shaped element 1 can be accommodated in a gathered manner. Recesses 46 of this type are illustrated purely by way of example in FIG. 16, and are used as a tension release in the event that the tube-shaped element 1 is unfolded prior to the securing of the gastrointestinal implant 17 on the pylorus 7 by means of the fastening means 3, 4, since in this case no tension that negatively affects the expansion of the fastening means 3, 4 can be exerted by the tube-shaped element 1 on the fastening means 3, 4.

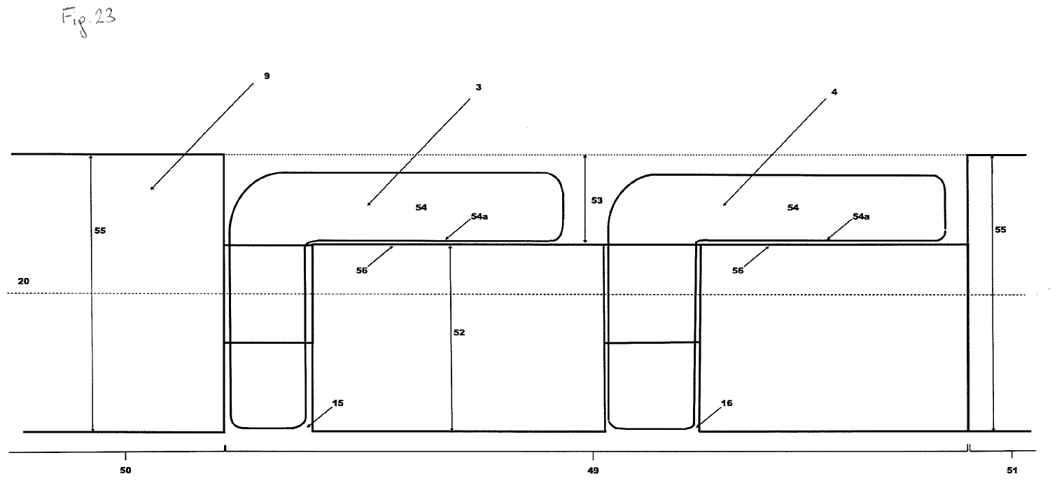

A particularly preferred embodiment of the inner supporting body 9 is illustrated in FIG. 23. A section 49 of the inner supporting body 9 is thereby embodied with a cross-sectional area 52 that is reduced compared to the regions 50, 51 adjacent thereto. The grooves 15, 16 are arranged in this section 49. The missing cross-sectional area 53 enables the accommodation of sections 54 of the fastening elements 3, 4 in this region, that is, one section 54 each of the fastening elements 3, 4 is folded in the direction of the longitudinal axis 20, as is illustrated in FIG. 23. The raising of the sections 54 is prevented by the outer vessel 8, which is not illustrated in this figure.

With a corresponding sizing of the reduced cross-sectional area 52 in relation to the cross sectional area 55 of the adjacent regions 50, 51 and the fastening elements 3, 4 as well as the grooves 15, 16, the fastening elements 3, 4 can be folded such that they are completely arranged within the cross-sectional area 55 of the adjacent regions 50, 51 and do not protrude past said area, whereby a particularly pronounced compression is possible.

As previously explained further above, the inner supporting body 9 has a circular cross section, that is, the cross-sectional areas 55 are preferably circular. The reduced cross-sectional area 52 can in principle have any desired shape, but is preferably embodied in the shape of a circle segment; however, it can also be produced by the removal of one or two, preferably opposing, cross-sectional surface(s) in the shape of a circle segment from a circular cross-sectional area. At any rate, in the latter case at least one very good support surface 56 is formed for the sections 54 or the faces 54a thereof.

It should be noted, solely for the sake of completeness, that the cross-sectional areas 55 of the sections 50, 51 can also be differently sized.

### Functional Principle of Invention

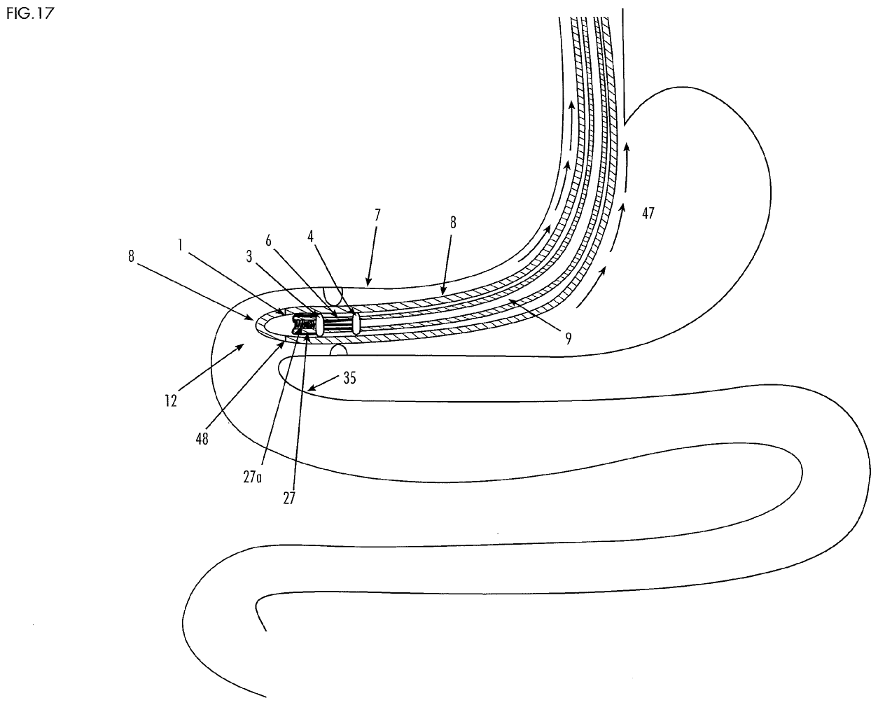

A detailed description of the functional principle of the invention will now follow below with the aid of FIGS. 17 through 21, which merely illustrate said principle in a schematic manner.

Initially, the positioning apparatus 18, as is illustrated by way of example in FIG. 12 or 13 with an applied gastrointestinal implant 17, is initially inserted transorally, that is via the mouth and the esophagus, into the gastrointestinal tract until the first fastening means 3 has assumed a position that is distal to the pylorus 7 and the second fastening means 4 has assumed a position that is proximal to the pylorus. This takes place by means of a guide wire (not shown) which is first introduced into the gastrointestinal tract accordingly by means of a gastroscope. Then, the outer vessel 8 is positioned in the intended position in the gastrointestinal tract via the guide wire, and the guide wire is removed again against the direction of insertion.

To determine the position of the positioning apparatus 18, it is advantageous if the outer vessel 8 and/or the inner supporting body 9 are provided with a marking 49 on their outsides that indicates to the physician performing the operation the insertion depth of the outer vessel 8 and the relative position of the inner supporting body 9 thereto, so that it can be precisely determined when the fastening means 3, 4 have reached their position distal from and proximal to the pylorus 7. Thus, for example, the distance between the patient's tooth row and the pylorus can be gastroscopically measured in advance.

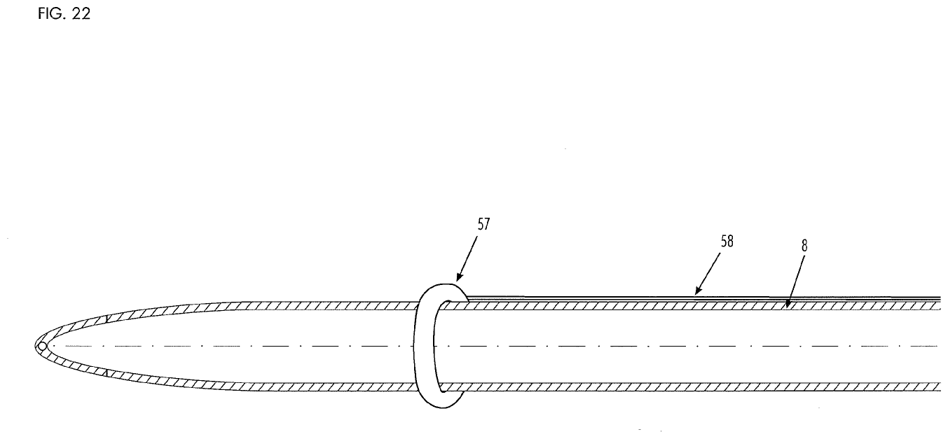

Alternatively or additionally, limit stops 57 (FIG. 22) can be provided which facilitate the insertion of the outer vessel 8 up to a defined position. The limit stop 57 can, for example, be embodied as an annular balloon that is permanently connected the outer vessel 8 and surrounds said vessel, and which can be filled with air via an insufflation tube 58 that runs on the surface of the outer vessel. When the balloon is in the stomach, it is inflated so that the positioning apparatus 18 can only continue to be advanced until the balloon strikes the pylorus. The position of the balloon on the outer vessel is thereby chosen such that, at this point in time, the first fastening means 3 is in a position in which the expansion thereof can be carried out.

As previously mentioned at the outset, it is advantageous if, at this point in time, the outer vessel 8 and the inner supporting body 9 are position-fixed relative to one another via securing means (not shown).

However, the following statements should be considered regardless of how the positioning apparatus 18 was placed in the correct position.

FIG. 17 shows the point in time after the transoral insertion at which the first fastening means 3 is located distally from the pylorus 7. At this point in time, the distal end 12 of the outer vessel 8 is still closed. The distal end region 1b of the tube-shaped element 1, which region can be located in a more or less invaginated state in the holding volume 27a of the aligning section 27, can also be closed or also open.

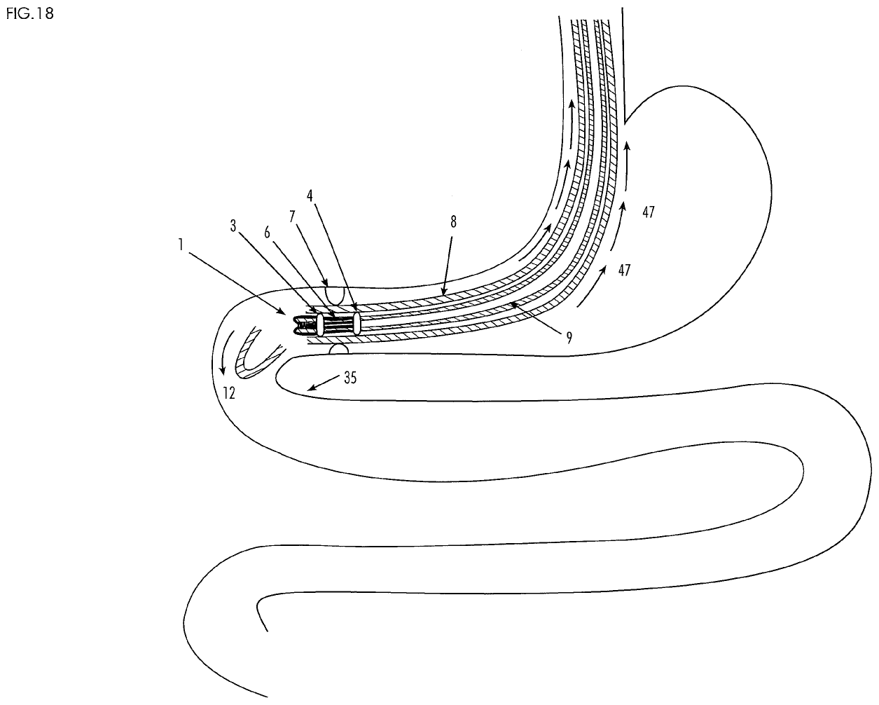

Subsequently, as a result of the slight retraction of the outer vessel 8 in the direction of the arrows 47 and the accompanying impact of the aligning section 27 on the distal end region 12, said region is separated from the remaining outer vessel 8, as is shown in FIG. 18, or else is simply opened, depending on whether the structural weakness 48 facilitating this runs across the entire circumference of the outer vessel 8 or only a section of the circumference. Alternatively, in the event that the distal end region 12 is constructed from absorbable material, time can be allowed to pass until said region dissolves.

During the retracting of the outer vessel 8, attention should in any case initially be paid that said vessel is not retracted over the position of the first fastening means 3 after the separation or opening of the distal end region 12.

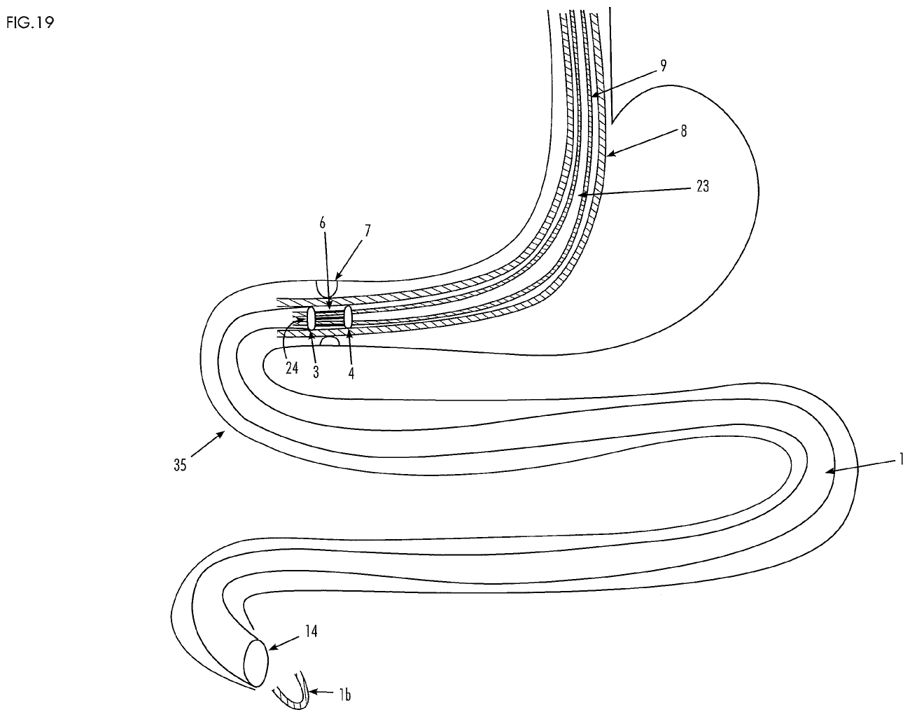

Subsequently, and as shown in FIG. 19, via a channel 23 running in the interior of the supporting body 9, which channel ends in an outlet opening 24 protruding in the holding volume 27a (see also FIGS. 9 and 10), a fluid, preferably CO2 or air, can be blown into said channel continuously or in a pulsating manner via the open end 1a of the tube-shaped element 1. Through the resulting increasing internal pressure, the section of the hose-shaped element 1 located in the holding volume 27a is transported out of said volume and, similarly to a balloon, inflated until it completely unfolds.

If the distal end 1b of the tube-shaped element 1 is closed, it must be detached from the remaining tube-shaped element 1 in order to ready said element for use. It can thereby be provided that the distal end 1b is made of absorbable material so the physician performing the operation only needs to wait for a defined period of time.

Alternatively, the tube-shaped element 1 can be provided in the end region 1b thereof with an opening (not shown) which is closed with a plug (not shown), or can be provided with a structural weakness 14 (see FIG. 1), for example in the form of a perforation or a thinned wall section, which separates the second end region 1b from the remaining tube-shaped element 1. Continuing to blow fluid into the tube-shaped element 1, which at this point in time is already completely unfolded, causes the internal pressure thereof to further rise, whereby the distal end region 1b detaches from the remaining tube-shaped element 1 at the structural weakness and can be naturally discharged.

The same thing applies for the embodiment with a plug, which can be formed from starch, for example, and also detaches from the opening as a result of the internal pressure, so that the tube-shaped element is then passable for the chyme.

In the event that the distal end of the tube-shaped element 1 is not closed, the latter is immediately ready for use after unfolding out of the holding volume 27a. Surprisingly, it became apparent that the folding or compressing of the tube-shaped element 1 in the holding volume 27a is sufficient to enable the unfolding by the insufflation of the fluid, even if the distal end 1b is not closed.

In one embodiment of the invention, it is provided that an X-ray strip is integrated in the tube-shaped element 1 in order to render the position of the tube-shaped element 1 and/or the complete unfolding thereof visible with X-rays.

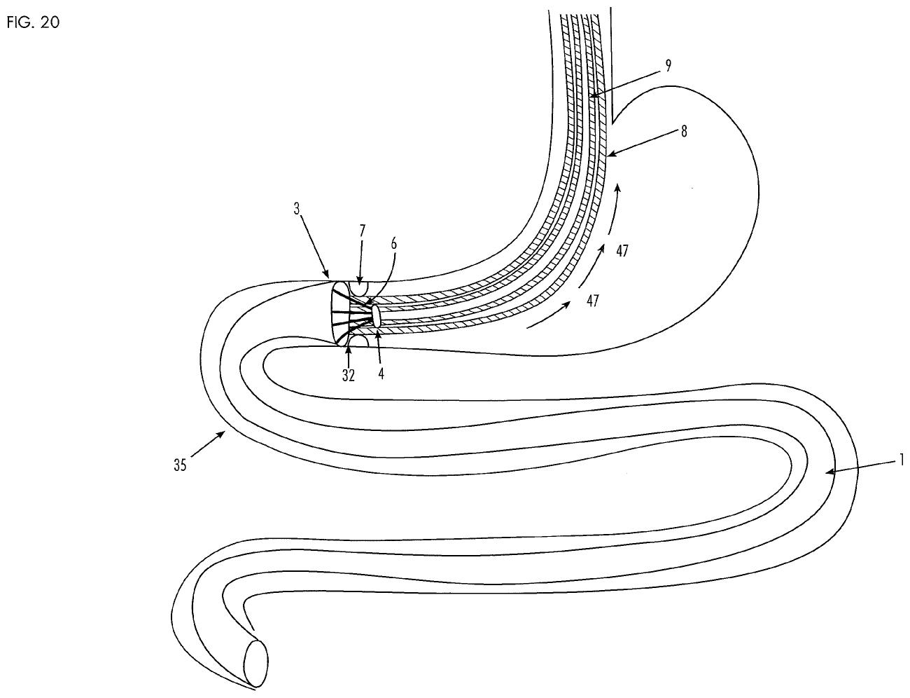

As a next step, the outer vessel 8 can then be retracted over the position of the first fastening means 3 in the direction of the arrows 47, as is illustrated in FIG. 20, so that the first fastening means 3 can be released from its mounting position 3a and an arboreal expansion of the first fastening means 3 occurs. Expediently, the inner supporting body 9 is provided with markings, so that it is easy to recognize how far the outer vessel 8 has already been retracted, and so that it is in turn possible to conclude therefrom whether a fastening means 3, 4 was already able to expand and, if so, which one.

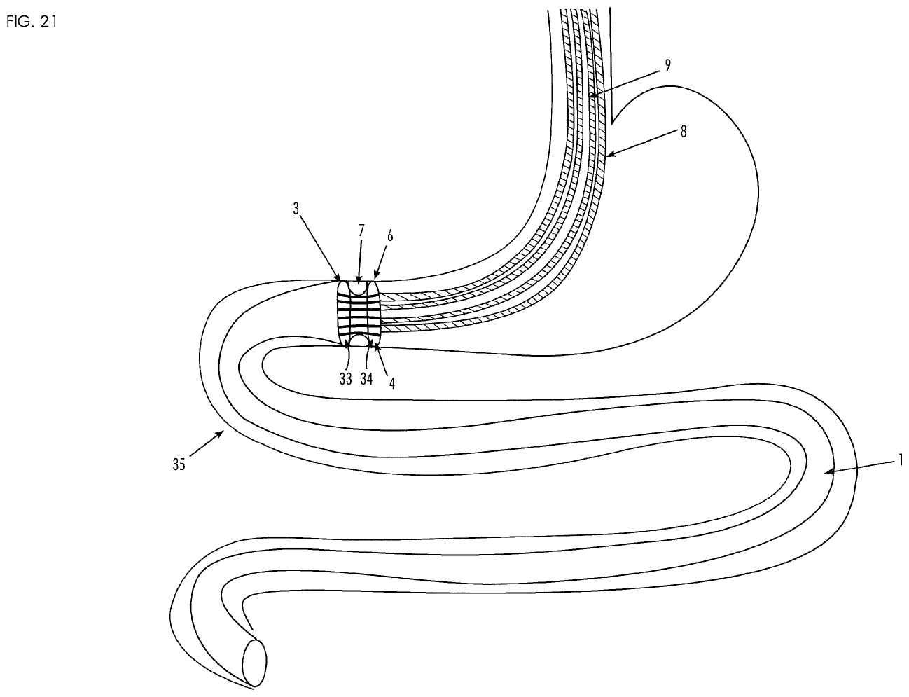

In the expanded form of the first fastening means 3, the outer vessel 8 can then be further retracted in the direction of the arrows 47, so that the second fastening means 4 also expands, as is illustrated in FIG. 21. At this point in time, the two fastening means 3, 4 are freely movable and are pulled towards one another by the connecting element 6, so that they bear with their contact surfaces 33, 34 against the distal and proximal front faces 31, 32 of the pylorus 7, respectively, and clamp it. The tube reserves accommodated in the recesses 46, where present, support this clamping as a result of the accompanying facilitated mobility of the fastening means 3, 4 in the axial direction. The gastrointestinal implant 17 is thus position-fixed, so that subsequently both the outer vessel 8 and also the inner supporting body 9 can be removed again via the esophagus. What remains is the gastrointestinal implant 17 secured on the pylorus as illustrated in FIG. 2.

At this junction, it should not go unmentioned that it is also possible to first secure the gastrointestinal implant 17 to the pylorus 7 using the fastening means 3, 4 and to only then effect the unfolding of the of the tube-shaped element 1 by blowing in a fluid via the channel 23.

### LIST OF REFERENCE NUMERALS

1. Tube-shaped element
1a. First (proximal) end of the tube-shaped element
1b. Second (distal) end of the tube-shaped element
2. Duodenum
3. First fastening means
3a. First mounting position
4. Second fastening means
4a. Second mounting position
5. Pyloric antrum
6. Connecting element
7. Pylorus
8. Outer vessel
8a. Option for accommodating a guide wire
9. Inner supporting body
10. Flow opening of the first fastening means
11. Flow opening of the second fastening means
12. Distal end region of the outer vessel
13. Proximal end region of the outer vessel
14. Structural weakness
15. First groove
16. Second groove
17. Gastrointestinal implant
18. Apparatus for positioning a gastrointestinal implant in the human gastrointestinal tract
19. Longitudinal axis of the outer vessel
20. Longitudinal axis of the inner supporting body
21. Rotationally symmetrical section of the inner vessel
22. An end region of the inner supporting body
23. Channel in the inner supporting body
24. Outlet opening of the channel
27. Aligning section for the tube-shaped element
27a. Holding volume
29. Magnetic section of the first fastening means
29a. Individual magnet in a magnetic section
30. Magnetic section of the second fastening means
30a. Individual magnet in a magnetic section
31. Proximal face of the pylorus
32. Distal face of the pylorus
33. Contact surface of the first fastening means
34. Contact surface of the second fastening means
35. Intestine
36. Attracting magnetic force
37. Repelling magnetic force
38. Interior volume of a supporting element
40. Coil spring element
41. Supporting element
41a. First end section of the supporting element
41b. Second end section of the supporting element
42. Supporting element
43. Coil spring element
44. Coil spring element
45. Coil spring element
46. Recesses on the surface of the inner supporting element
47. Directional arrows
48. Structural weakness of the outer vessel
49. Section of the inner supporting element with reduced cross-sectional area
50. Section of the inner supporting element adjacent to 49
51. Section of the inner supporting element adjacent to 49
52. Reduced cross-sectional area
53. Missing cross-sectional area
54. Section of the fastening means 3, 4
54a. Faces of the sections 54
55. Cross-sectional area of the adjacent sections 51, 52
56. Support surface
57. Limit stop
58. Insufflation tube

1.-29. (canceled)

30. A positioning apparatus for positioning a gastrointestinal implant with two essentially annular fasteners and a tube-shaped element in a human gastrointestinal tract, comprising:
an outer vessel comprising a first longitudinal axis;
an inner supporting body, which is positionable within the outer vessel, comprising a second longitudinal axis, wherein the inner supporting body enables a sliding-on of the annular fasteners;
a first mounting position for the first fastener, which is arranged between the outer vessel and the inner supporting body; and
a second mounting position for the second fastener, which is arranged between the outer vessel and the inner supporting body,
wherein the outer vessel is removable from the inner supporting body, preferably axially detachable in a direction of the second longitudinal axis
wherein the first and second mounting positions each comprise an outer limit formed by the outer vessel and an inner limit formed by the inner supporting body
in order to clamp the fasteners in their mounting positions, whereby the distance between the mounting positions corresponds at least the thickness of the pylorus.

31. The positioning apparatus according to claim 30, wherein the inner supporting body comprises a surface having two grooves for at least partially accommodating the annular fasteners, respectively, and
wherein a minimum distance between the grooves is at least one of:
3 mm-70 mm, or
5 mm-30 mm.

32. The positioning apparatus according to claim 30, wherein the outer vessel is embodied in a sleeve shape and a distal end region of the outer vessel is embodied to be closed and at least one of removable or openable.

33. The positioning apparatus according to claim 32, wherein the outer vessel comprises a structural weakness, preferably in the form of a predetermined breaking point, which enables the removal or opening of the distal end region from a remaining part of the outer vessel.

34. The positioning apparatus according to claim 32, wherein an end region of the inner supporting body located closer to the distal end region of the outer vessel comprises an aligning section for a section of the tube-shaped element, which comprises a preferably cup-shaped holding volume in which a portion of the tube-shaped element can be accommodated in an invaginated manner.

35. The apparatus according to claim 34, wherein the inner supporting body comprises a through channel having an outlet opening in the aligning section.

36. The apparatus according to claim 30, wherein the inner supporting body comprises a section have a reduced cross-section compared to adjacent sections, and the reduced cross-section section comprises at least one support surface running in the direction of the second longitudinal axis for supporting frontal sections of the annular fasteners.

37. A gastrointestinal implant system comprising:
a gastrointestinal implant that comprises:
a tube-shaped element, embodied to transport chyme through at least one section of a human intestine;
a first fastener, which is connected to the tube-shaped element, comprising a first pass-through opening, the first fastener being embodied to be positionable in a section of a duodenum adjacent to a pylorus;
a second fastener, which is connected to at least one of the tube-shaped element or to the first fastener, comprising a second pass-through opening, the second fastener being embodied to be positioned in a gastric antrum; and
a connecting element that connects the at least one of the first fastener or the tube-shaped element to the second fastener; and
an apparatus that is configured to position the gastrointestinal implant in a human gastrointestinal tract and comprises:
an outer vessel comprising a first longitudinal axis;
an inner supporting body, which is removably positionable in the outer vessel, comprising a second longitudinal axis;
a first mounting position for the first fastener, which first mounting position is positioned between the outer vessel and the inner supporting body; and
a second mounting position for the second fastener, which second mounting position is positioned between the outer vessel and the inner supporting body,
wherein the first and second fasteners are slidable onto the inner supporting body to be position-fixed in the first and second mounting positions, respectively, and to at least one of be compressed to a first size or assume a first position, and
wherein the outer vessel is removable from the inner supporting body, preferably axially detachable in a direction of the second longitudinal axis, and
wherein, the inner supporting body, when removed from the outer vessel, the first and second fasteners to at least one of expand to a second size or assume a position different from the first position.

38. The system according to claim 37, wherein the connecting element is embodied to bias the first and second fasteners toward one another so that, with respective first and second contact surfaces of the first and second fasteners being respectively positioned in the duodenum and in the gastric antrum, with the pylorus positioned therebetween, the first and second fasteners are not lifted off of the pylorus.

39. The system according to claim 37, wherein the outer vessel is embodied in a sleeve shape and a distal end region of the outer vessel is embodied to be closed and at least one of removable or openable.

40. The system according to claim 37, wherein the tube-shaped element comprises a second end region that is embodied to be closed and removable, preferably made of a material that can be absorbed in the intestine.

41. The system according to claim 37, wherein the first mounting position for the first fastener and the second mounting position for the second fastener comprise an outer limit formed by the outer vessel and an inner limit formed by the inner supporting body and the distance between the limits is thereby selected such that the two fasteners are clamped between the outer vessel and the inner supporting body.

42. The system according to claim 37, wherein the inner supporting body comprises a surface having two grooves for at least partially accommodating the annular fasteners, respectively, and
wherein a minimum distance between the grooves is at least one of:
3 mm-70 mm, or
5 mm-30 mm.

43. The system according to claim 37, wherein the fasteners are folded down in direction of the longitudinal axes.

44. The system according to claim 43, wherein a section of the inner supporting body is thereby embodied with a cross-sectional area that is reduced compared to the regions adjacent thereto and the folded down fasteners are positioned within this section.

45. The system according to claim 37, wherein an end region of the inner supporting body located closer to a distal end region of the outer vessel comprises an aligning section for a section of the tube-shaped element, which comprises a preferably cup-shaped holding volume.

46. The system according to claim 45, wherein the inner supporting body comprises, at least in sections, a channel having an outlet opening in the aligning section.

47. The system according to claim 45, wherein the tube-shaped element comprises a region which is pulled over the aligning section that is preferably embodied in a cup shape.

48. The system according to claim 47, wherein the region of the tube-shaped element which is pulled over the aligning section is in flow connection with the outlet opening.

49. The system according to claim 47, wherein the inner supporting body further comprises a cup-shaped aligning section, and wherein a section of the tube-shaped element not pulled over the aligning section is invaginated, at least in sections, preferably completely, in the cup-shaped holding volume and is arranged therein preferably in a folded manner.

50. A method for inserting a gastrointestinal implant in a part of an intestine, the gastrointestinal implant including a tube-shaped element configured to transport chyme through at least a section of the intestine, a first compressible fastener, connectable to the tube-shaped element, having a first pass-through opening, a second compressible fastener, connectable to at least one of the tube-shaped element or to the first fastener, having a second pass-through opening, and a connecting element that connects the at least one of the first fastener or the tube-shaped element to the second fastener, the method comprising:
sliding the first and second compressible fasteners onto an insertion end of an inner supporting body;
inserting the inner supporting body into an outer vessel, wherein the first and second compressible fasteners are positioned between the inner supporting body and the outer vessel in respective first and second mounting positions;
guiding the outer vessel and inserted inner supporting body to position the insertion end within a pylorus between a duodenum and a gastric antrum;
removing the outer vessel from insertion end so a contact surface of the first compressible fastener is in the duodenum and a contact surface of the second compressible fastener is in the gastric antrum; and
biasing the first and second compressible fasteners toward each another to clamp the pylorus between the first and second compressible fasteners.

51. The method according to claim 50, wherein, before or during sliding the first and second compressible fasteners onto the insertion end, compressing the compressible first and second fasteners, whereby the compressible first and second fasteners in the respective first and second mounting positions are held in a compressed state.

52. The method according to claim 51, wherein, when removing the outer vessel from the insertion end, the compressible first and second fasteners expand to an expanded state.

53. The method according to claim 50, wherein the inner supporting body includes a holding volume, and the method further comprises folding and inserting a portion of the tube-shaped element into the holding volume

54. The method according to claim 50, wherein the tube-shaped element comprises an open end and the inner supporting body includes a fluid channel communicating with the open end, the method further comprising supplying a fluid through the fluid channel to expand the tube shaped element.

55. The method according to claim 50, wherein the outer vessel has a closed end to facilitate the guiding of the insertion end within the pylorus, and the method further comprises at least one of opening or removing the closed end after the insertion end is within the pylorus.
